(19) United States
(12) Patent Application Publication
(10) Pub. No.: US 2019/0380747 A1
(43) Pub. Date: Dec. 19, 2019

(54) AXIALLY PRECISE SCREWDRIVER

(71) Applicant: Aesculap AG, Tuttlingen (DE)

(72) Inventors: Kay FISCHER, Tuttlingen (DE); Stephan LINDNER, Wurmlingen (DE)

(21) Appl. No.: 16/480,559

(22) PCT Filed: Jan. 25, 2018
(86) PCT No.: PCT/EP2018/051868

§ 371 Date: Jul. 24, 2019

(30) Foreign Application Priority Data
Jan. 25, 2017 (DE) 10 2017 101 348.2

(51) Int. Cl. A61B 17/70; B25B 15/00; B25B 23/10

(52) U.S. Cl. CPC A61B 17/7082; A61B 17/7034; B25B 23/108; B25B 15/004

(57) ABSTRACT

An implantation set for implanting a screw in a human or animal body includes a tool and a screw designed to receive the tool. The screw has a tool receiving region into which or onto which a torque transmission region of the tool can be or is inserted or placed to transmit torque. At least one inclined insertion region is formed on the tool receiving region to guide the tool during insertion of the screw. The tool forms at least one centering surface that is matched to the inclined insertion region such that when the centering surface comes into contact with the inclined insertion region, a form fit and force fit is created.

### CROSS REFERENCE TO RELATED APPLICATION(S)

This application is the United States national phase entry of International Application No. PCT/EP2018/051868, filed Jan. 25, 2018, which claims the benefit of priority of German Application No. 10 2017 101 348.2, filed Jan. 25, 2017. The contents of International Application No. PCT/EP2018/051868 and German Application No. 10 2017 101 348.2 are incorporated by reference herein in their entireties.

### FIELD

The present invention relates to an implantation set for implanting a medical (bone) screw in a human or animal body, comprising a tool and a screw designed to receive the tool. Furthermore, the present invention relates to a screw and an appropriately shaped tool which are intended and designed to form the implantation set according to the invention. The screw or its screw head has a tool holding/insertion area, or more precisely an inclined insertion region, which is formed proximal to a torque receiving/insertion region in the screw head. The inclined insertion region is in faun-fitting and force-fitting contact with a centering surface of the tool. The centering surface of the tool is formed proximal relative to a torque transmission portion of the tool, which is configured and adapted to be inserted into the torque-receiving/introduction portion in the screw head.

### BACKGROUND

The prior art knows screws for implantation in human and animal bodies, such as pedicle screws, for example. Suitable tools are also known, which can be inserted into a blind hole with a multi-groove profile (multi-lobe profile) in a screw head of such a screw or placed thereon to apply a torque to the screw.

Form-fit connections between the screw and a power transmission tool are conventionally designed in the nature of a cross-slot, hexagonal or Torx configuration. Such cross-slot, hexagonal or Torx designs of the coupling sections of the two components are widespread and well known. The term “Torx” is used to describe a type of screw that has a multi-lobe drive profile design. It resembles a Star of David with rounded tips and grooves as well as rounded corners. The tool engagement between tool and screw is able to transmit high torques without damaging the screw head. Such a Torx is also understood as a “six-lobe”, for example.

It is noteworthy that with such Torx profiles, it is easier to attach/detach the tool and improved force transmission is possible as compared to slotted or cross-head screws. Due to the vertical drive/torque transmission surfaces of a multi-groove profile or Torx profile, no or only a barely increased pressure force in the axial direction of the tool is required when tightening, and no or hardly any back thrust forces occur, thus preventing the so-called “cam-out”. It is also noteworthy that torsional forces are not applied at certain points, as is usual with Allen key systems, but in the middle and over a large area on the flanks of the individual webs (extending in the axial direction of the tool) forming the drive surfaces. The result is that the system consisting of a male and a female element is significantly less subject to wear. Especially in surgery and dental prostheses, the use of screws together with a corresponding tool mount and a tool adapted to it is therefore an obvious choice. This allows bone fracture treatments to be carried out more precisely and with less problems. Implants can also be placed more easily.

The term “Torx” here refers to both internal and external profile types marketed under the federally registered trademark TORX®. These include T, E and TR variants, as well as variants marketed under the federally registered trademarks TORX+® and HEXSTIX®.

In almost all areas, in particular also in surgery, connections are used which rely on form-fit locking. Connections of this type are used in spinal surgery, e.g. to screw in bone screws, to tighten locking screws or to secure similar moving parts. While the Allen system was the first choice up to now, the Torx design is increasingly gaining acceptance for the reasons mentioned above.

In medical applications, the advantages of Allen and Torx geometries, where the cylindrical part predominates, are particularly evident. As a result, the screwdriver does not have to be pressed axially into the screw head during screwing in, as is the case with crosshead geometries, but only has to follow the screw-in depth without force.

Screws used in the area of the spine, e.g. pedicle screws, must often be coupled “in situ” at depth or without any visual contact. This is particularly the case with screwdrivers marketed by Aesculap AG under the federally registered trademarks S4® and ENNOVATE®, in which the bone screw or its screw head is enclosed by a so-called “tulip”, which obstructs the view, so that the tool has to be coupled or inserted into the tool mount on the screw head side by feeling. Tactile feedback is therefore decisive for successful insertion (successful insertion or placing of the tool into or on the screw).

Bone cement is used, for example, in patients whose bone quality is not sufficient to firmly anchor a screw. For this purpose, a hole is drilled in a vertebra of the spinal column, filled with bone cement or another stabilizing substance, and a pedicle screw is then inserted. Bone cement is usually a two-component adhesive that can cure to result in acrylic glass, among other things.

DE 10 2013 105 744 A1 according to Haas et al. discloses an implantation screw and a tool of an implantation set with constrained relative rotation. More precisely, an implantation set is disclosed in which the screw has a tool receiving/torque transmission region adjoined by at least one inclined region for guiding/inserting the tool into/in the tool receiving/torque transmission region (during inserting/introducing) into the screw.

WO 2011/043799 A1 according to Lange et al. discloses a fixing system and a screwdriver for use with the same.

The screws used in the area of the spine, e.g. pedicle screws, are screwed into the spine using a screwdriver. The point of connection between the pedicle screw and the screwdriver is usually only provided by a multi-lobe drive profile design in accordance with the Torx geometry above, to be more precise by its drive surfaces extending perpendicularly/parallel to the tool axis. A connection between screw and screwdriver that is not axially precise can result in loosening or, in the worst case, pedicle fracture as a result of screwing in or unscrewing the bone screw. There is also the problem that the tightness against bone cement and the torsional strength are not reliably secured when the screwdriver is placed/inserted. Furthermore, the alignment between screwdriver and screw is not (sufficiently) stabilized with an effective torque, which makes the alignment of the screwdriver axis to the axis of the screw unstable. Re-placing or re-insertion of the tool into the screw is often difficult, especially when the screw has already been implanted.

### SUMMARY

It is therefore the object of the invention to provide a (fixed) connection between screwdriver and screw (pedicle screw) that can transmit the highest possible torque. Another advantageous object of the invention is that the screw has no play relative to the screwdriver and can be aligned with the axis of the screwdriver in axially precise manner, whereby the axis accuracy is maintained even under the influence of greater lever forces. A further object of the invention is that when the screwdriver is again coupled to the screw, especially in the intraoperative (in situ) field, the axial accuracy between the screw and the screwdriver can be restored by firmly connecting the parts. In other words, there will be a compulsory alignment of the screwdriver axis with the screw axis when the screwdriver is connected/coupled to the screw. Furthermore, it should be prevented that bone cement can escape in an uncontrolled way.

This object is achieved by an implantation set and screw as described herein.

The basic idea of the present invention is therefore to form a screw, preferably a bone screw, more preferably a pedicle screw with a profiled, blind hole-type tool mount in its screw head region, comprising a (distal) torque introduction portion, preferably in Torx/multi-groove/Allen shape, the (proximal) outer circumferential edge of which is formed into an inclined insertion region. According to the invention, this inclined insertion region represents/forms a continuation of the torque introduction portion, preferably in Torx/multi-groove/Allen shape, wherein the webs and grooves which are formed thereby do not extend axially parallel in the inclined insertion region (directly proximally adjoining the torque introduction portion) as is the case in the torque introduction portion but at an angle greater than 0° relative to the (screw) axis in the direction proximally obliquely outwards in extension to the axial webs and axial grooves in the torque introduction portion. The grooves/indentations in the inclined insertion region are preferably cup-shaped or spherical and each form funnel-shaped or sickle-shaped contours in plan view of the tool mount, which are therefore preferably arranged on a circular path around the blind hole axis at preferably the same circumferential distance.

The outer radius of the grooves/indentations in the inclined insertion region is dimensioned such that the outer radii of respectively two grooves/indentations adjacent to each other in the circumferential direction intersect in the inclined insertion region.

The associated/matching (screw-in) tool has a shank, at the distal end region of which a torque transmission portion is preferably formed or is or can be arranged in Torx/multi-groove/Allen shape, and which is configured and/or adapted to be inserted into the screw-side torque introduction portion (in a form-fitting fashion for torque transmission). The torque transmission portion of the tool can preferably be chamfered at its distal end in the manner of a truncated cone in such a way that the tool tip or the web-shaped projections of the Torx/multi-groove/Allen profile which are fottned there and taper in the manner of a truncated cone are centered in the blind hole-like tool mount and the tool shank is aligned coaxially to the screw axis (blind hole axis) by sliding engagement with the screw-side inclined insertion region or the (spherical-head-shaped) grooves/indentations formed there.

Since sliding insertion of the tool-side torque transmission portion into the screw-side torque introduction portion within the blind hole-type tool mount must be ensured, there is inevitably play between the grooves and webs in the tool mount and the webs and grooves on the tool (shank), which can cause a tilting tendency of the tool shank in the blind hole-type tool mount.

For this reason, the tool shank at the proximal (rear) end of its torque transmission portion forms a radially protruding shaft/shank shoulder into which the profile of the screw-side inclined insertion region is machined in a mirror-inverted/complementary manner. This means that in the tool-side shaft shoulder, a number of (axially) distally extending, preferably spherical projections are formed in the screw-side inclined insertion region, corresponding to (matching with) the spherical-head-shaped recesses/indentations in the screw-side inclined insertion region, which, in accordance with the invention, when the tool-side torque transmission portion is completely inserted in the screw-side torque introduction portion/region into the spherical calotte-shaped recesses/indentations in the screw-side inclined insertion region and thereby radially clamp the tool shank in the blind hole-like tool mount of the screw. In addition, there is a form fit in the circumferential direction. In this way, the tilt tendency described above can be reduced or avoided.

In other words, the area of the screw which is intended and adapted to accommodate a screwdriver, i.e. that area of the screw which has a relative recess (blind hole) in the screw head, fits exactly to a corresponding screwdriver head. Accordingly, the present invention describes a screwdriver which, in combination with mono- or polyaxial pedicle screws, for example, produces an axially precise and torsion-resistant as well as cement-tight joint, whereby the screw-side inclined insertion region comes into contact with the correspondingly shaped shaft shoulder on the tool side in a substantially sealed manner. Due to this special design, in-situ reconnection is also possible in a simple and safe way.

The present invention thus relates to an implantation set for implanting a screw in a human or animal body, having a (screw-in) tool and a screw designed for receiving the tool, for which purpose the screw has a tool receiving region/portion in its screw head, into which (or onto which) a torque transmission region/portion of the tool can be inserted/attached or is inserted/attached for torque transmission, wherein at least one inclined insertion region/portion for guiding/aligning the tool (tool shank) during insertion into the screw being formed on the tool receiving region at its proximal edge (insertion opening). In accordance with the invention, the tool forms at least one (annular) centering surface (shaft/shank shoulder) which delimits the torque transmission region proximally (at a distance from the tool tip) and is matched to the inclined insertion region in such a way that, when the centering surface comes into contact with (abuts on) the inclined insertion region, a form fit and force fit is produced between the inclined insertion region and the centering surface in addition to the torque transmission region/portion.

The ability (effect) to transmit the torsional moment between the screwdriver or screwdriver shaft and the screw or bone screw is thus additionally enhanced (increased) by the form fit and force fit between the centering surface and the inclined insertion region. Having a form-fit and/or force-fit engagement between the front-end countersink (inclined insertion region) of the screw and the complementary centering surface (shaft/shank shoulder with radially and axially projecting, preferably semi-spherical projections) on the screwdriver, torsional transmission takes place via a cross-section/diameter which is larger than that of the multi-lobe (multi-groove profile) disposed below/distal. This results in a higher torsional strength. Due to the complementary fit between the tool-side centering surface and the screw-side countersink or the inclined insertion region, a higher degree of sealing or tightness is achieved for the safe application of bone cement.

The screw according to the invention is preferably a pedicle screw comprising
a bone screw having a screw shaft and the screw head defined above,
a body or tulip, which is held movably on the screw head and has an internal thread, preferably for receiving a grub screw (corresponds to a polyaxial pedicle screw), and optionally
an insert (pad for tensioning the screw head and tulip) and the grub screw.

The screw-in tool according to the invention preferably comprises
a tool shank with a distal torque transmission portion and a shaft/shank shoulder (centering surface) arranged proximally thereto in accordance with the above definitions,
a counterholder fixed/connectable to the tool shank in the form of a shaft shoulder/shaft ring, preferably elliptical in cross-section and proximal to the centering surface, which is configured and/or adapted to be inserted into a longitudinal gap of the tulip in order to also hold the tulip in rotationally manner when the tool shank is held in rotational manner, and
a sleeve-shaped threaded part having an axial through-hole into which the tool shank is inserted to be rotatably and axially movably therein, such that the sleeve-shaped threaded part is arranged proximally to the counterholder, the sleeve-shaped threaded part having at its distal end portion an external thread which is configured and/or adapted to engage with the internal thread of the body/tulip, and the sleeve-shaped threaded part preferably having at its proximal end portion an engagement profile which can be brought into engagement with a torque-impacting tool/means, e.g. a wrench to (temporarily) drive in the sleeve-shaped threaded part into the body/tulip while the body/tulip is rotationally held by the tool shank and counterholder.

Advantageous embodiments are described below.

In an advantageous embodiment, torque transmission regions, more precisely axially extending ribs or webs, are formed on a cylindrical side surface of the tool and extend over a distally adjoining, frustoconical side surface section up to a tip of the tool. In other words, the frustoconical side surface or conical tip portion of the screwdriver has a frustoconical or conical multi-lobe. This means that when the threaded part is tightened, the conical tip section (the screwdriver tip) cannot rest bluntly on the proximal end face of the screw, i.e. on the side where the countersink/blind hole on the screw head is located, but is automatically aligned in conjunction with the front-end countersink or its inclined insertion region on the screw such that the multi-lobe geometry of the screwdriver shaft is aligned with the multi-lobe geometry of the screw.

As explained above and according to a preferred further development, the tool has the screwdriver shaft where the threaded part is attached/disposed, which has an external thread at its distal end that can be screwed into the internal thread of the body (tulip) which is movable relative to the screw shaft/screw head and is arranged on the screw. The (sleeve-shaped) threaded part is arranged on the shaft of the screwdriver above the counterholder and encloses the screwdriver with its cylindrical hollow shape. The external thread is located on the side of the threaded part facing the screw and thus also the counterholder. By tightening the threaded part, an axially parallel alignment of the screwdriver relative to the screw can be forced. In other words, the longitudinal axis of the screw and the longitudinal axis of the screwdriver are adjusted and are thus parallel to each other (co-axial).

In another advantageous embodiment, the screwdriver has its shaft provided with the counterholder which can be received in the body. The counterholder, due to being received in the body and owing to its shape, allows that the body cannot turn when screwing in the threaded part. The design of the counterholder is therefore provided and adapted to be received by the body. Due to the fact that the body laterally has openings/longitudinal slits on two diametrically opposite sides, it is also possible to insert the screwdriver shaft into the body at an angle to the screw axis. This additional degree of freedom represents a considerable simplification when reconnecting the screwdriver.

In a further advantageous embodiment, the body comprises the insert or received it, with the body forming an inner bore which is intended and adapted for receiving the screwdriver shaft, or more precisely for receiving the shaft of the screwdriver underneath the counterholder. The inside of the body has a shell-like shape on the side facing the tool, so that the tool tip is guided toward the inner bore when the tool is inserted.

In a further advantageous embodiment, the configuration according to the invention of screw, body, screwdriver, possibly insert and threaded part is designed in such a way that the threaded part can only engage in the internal thread of the body when the screwdriver tip is aligned in such a way that the multi-lobe geometry of the screwdriver and the screw have aligned themselves with one another (i.e. the torque transmission portion of the tool is already introduced in the torque introduction portion of the screw). This prevents incorrect coupling of the screwdriver in a way that is not aligned with the screw axis.

The implantation set described above allows the in-situ repositioning of the screwdriver on the screw by actively tightening the threaded part with the body so that the screwdriver and the screw are clamped relative to each other. This causes the screwdriver to align with the screw. The screwdriver can also be put on if the screwdriver is not actively brought into axial alignment with the body. The body axis is automatically aligned with the screwdriver axis when the screwdriver is put on. This automatic alignment simplifies the placement of the tool on the screw in general, but especially the repositioning of the tool on the screw, since an active/non-automatic alignment of the screwdriver axis to the body axis may be difficult during surgery.

The invention ensures a simple and safe reconnection of the screwdriver to the screw, preferably to a pedicle screw. Alignment of the screwdriver relative to the screw is made possible without the need to align the screwdriver with the axis of the body right at the beginning of the recoupling process. For in-situ recoupling, the screwdriver tip is inserted into the pedicle screw/its blind hole. The conical tip centers by means of the bore of the insert. The tool's counterholder must be aligned such that it can be inserted into at least one rod receiving groove/longitudinal slot of the body. The external thread of the sleeve-like threaded part cannot yet be brought into engagement with the internal thread of the body when the tool is inserted obliquely. The screwdriver is now advanced to/inserted in the pedicle screw. The centering tip of the tool and the fit between the shank portion/torque transmission portion below/distal to the counterholder and the inner bore of the insert align the body relative to the screwdriver. If the tool geometry of the screwdriver is aligned with the tool receiving geometry of the screw in the rotational direction, the axis of the body is also aligned with the axis of the threaded part to such an extent that the threaded part can be screwed into the internal thread of the body. The distal cone section of the tool is complementary to the spherical calotte-shaped grooves/countersinks/indentations in the inclined insertion region of the screw, so that this leads to a simple alignment of the tool geometry. When tightening the threaded part in the body/tulip, a fixed alignment of the axes is achieved by pressing the spherical projections on the tool shaft-side shank shoulder into the spherical/calotte-shaped grooves/countersinks/indentations in the inclined insertion region of the screw. This results in a form-fitting and force-fitting connection of the spherical projections with the spherical/calotte-shaped grooves/countersinks/indentations. This also ensures a cement-tight connection between the screwdriver and the screw.

In summary, there are many advantages offered by the design, according to the invention, of the implantation set. A very stable connection between screw and screwdriver is achieved. The axis of the screwdriver is reliably aligned with the axis of the screw. The in-situ recoupling of the screwdriver to the screw is considerably simplified. A high torque resistance of the joint is achieved by means of the form-fit and force-fit connection. The joint between screwdriver and screw is tight enough to prevent leakage during the application of bone cement. The effect of aligning the axis of the screwdriver with the axis of the screw is enhanced by the effect of the screw-in torque. The present connection also offers a cost-effective design due to the overall machinability of the working end with only one tool, such as a die-milling cutter with a diameter of preferably 0.8 mm.

### BRIEF DESCRIPTION OF THE DRAWING FIGURES

The invention is explained in more detail below using a preferred exemplary embodiment with reference to the accompanying Figures wherein:

FIG. 1 shows the components of an implantation set according to the invention in engagement with each other;

FIG. 2 shows a tool head of a tool according to the present invention;

FIG. 3 shows a threaded part arranged on the tool, in the present case in the form of a screwdriver;

FIG. 4 shows a body/a tulip attached to a screw, in the present case a pedicle screw;

FIG. 5 shows an insert arranged in the body/the tulip;

FIG. 6 shows a screw head of the screw/pedicle screw to be implanted, in the present case with a polyaxial design;

FIG. 7 shows the screw head of FIG. 6 in a detailed view; and

FIG. 8 is a side view of the tool in the state when clamped in the screw, arranged in the insert and the body.

### DETAILED DESCRIPTION

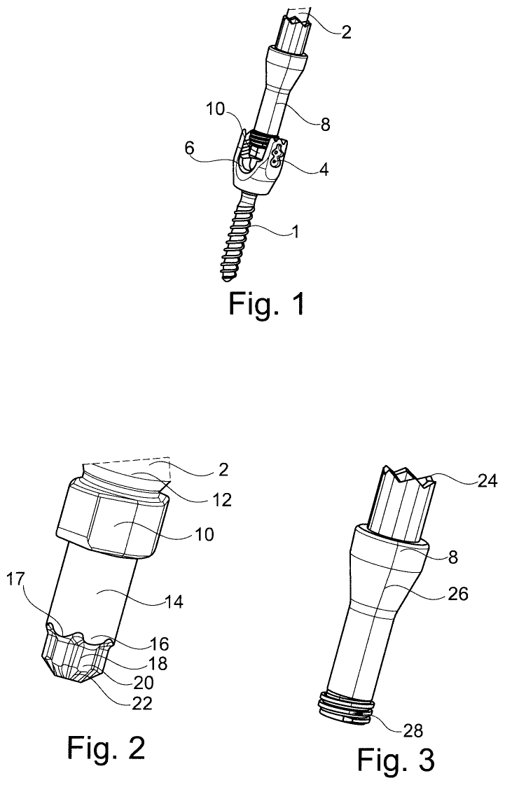

In FIG. 1, the components of an implantation set are shown in engagement with each other according to the preferred exemplary embodiment of the present invention. A screw, in the present case a pedicle screw 1, is in engagement with a (screw-in) tool/screwdriver 2. A movable body (tulip) 4 is placed axially over the screw 1 at its proximal screw head, which optionally has an insert/inlay 6 which is held so as to be axially movable relative to the body 4.

Radially above the tool 2 or its tool shank 12, a threaded part/threaded sleeve 8 is placed from the proximal side in the direction towards the distal tool tip/shank tip of the tool 2, which rests axially on a counterholder (approximately oval/elliptical shaft ring) 10 which is arranged/formed on the screwdriver/tool shank at a specific/determinable axial position (axially fixed).

FIG. 2 shows the head/tip portion/tool head of the tool 2 in a detailed illustration.

The tool head is formed on the preferably cylindrical screwdriver shank 12, which extends from a handle (not shown) to the counterholder 10. The counterholder 10 has a preferably hammer-shaped (oval/elliptical) cross-section or the design of a wing nut, with two diametrically opposite sides/wings projecting radially beyond the circumference of the cylindrical shank 12 extending above the counterholder 10 (proximally to it). A shaft portion 14 below/distally relative to the counterholder 10 and extending the tool shaft 12 in distal direction is smaller in diameter than the shaft 12 above (proximal to) the counterholder 10. The shaft portion 14 below (distal to) the counterholder 10 merges distally into a centering surface portion 16 or shaft/shank shoulder for centering the tool shaft 14 with respect to the screw 1, for additional torsion transmission and for sealing. The centering surface portion 16 merges distally into a multi-lobe profile/torque transmission portion 18 having axially extending, radially projecting and circumferentially spaced ribs/webs/teeth 20 on its cylindrical outer surface. The transition between the centering surface portion 16 and the multi-lobe portion 18 is preferably formed by rounded geometries (centering surfaces/protrusions 17) arranged in series in the circumferential direction.

In other words, a number of circumferentially spaced projections 17 are formed on the shaft/shank shoulder 16, each extending axially and radially and forming circumferentially spaced grooves/depressions in the axial and radial directions between them. The projections 17 and grooves on the shaft/shank shoulder 16 are arranged in axial extension to the ribs/webs 20 and grooves in the torque transmission portion 18.

The circumferential surface of the frustoconical tool end portion 22, or centering tip, also forms a multi-lobe geometry (in extension to the multi-lobe geometry in torque transmission portion 18). The primary torque transmission region/portion 18 is formed by the (axially extending) ribs 20 of the multi-lobe unit. The ribs 20 thus extend from the cylindrical peripheral surface of the multi-lobe unit across the peripheral surface of the shaft truncated cone 22 distally adjacent to the torque transmission region 18 to the tool shank tip which is flattened at the end face.

From FIG. 2 it can also be seen that the centering surfaces/protrusions in the area of the shaft/shank shoulder 16 and the ribs 20 align axially with one another (in extension), the individual centering surfaces/protrusions 17 in the shaft/shank shoulder 16 each being formed as semi-conical axial-radial projections above the ribs 20 and being spaced apart in the circumferential direction by cup-shaped/calotte-shaped recesses/grooves which are formed in the shaft/shank shoulder 16 and which are also arranged in extension to the grooves in the torque transmission portion 18.

FIG. 3 shows the sleeve-shaped threaded part 8 in detail, which can be/is arranged on the screwdriver 2, i.e. on the tool shank 12, according to the invention so as to be relatively movable thereto. The threaded part 8 has a continuous hole (axial through-hole) 24 for rotationally and axially displaceably receiving the screwdriver shank 12, whereby the hole diameter is complementary to the outside diameter of the shank 12 above the counterholder 10 for an essentially backlash-free support. The threaded part 8 rests axially on the counterholder 10 of the tool 2. On the axial end side of the threaded part 8 facing the counterholder 10, i.e. the distal end of the threaded part 8, an external thread 28 is formed underneath a preferably externally smooth sleeve portion 26. In addition, an axial prong-type profile is worked out at the upper, proximal end of threaded part 8 in order to be able to apply a torque to the threaded part 8 using an appropriate tool (e.g. a wrench, not shown).

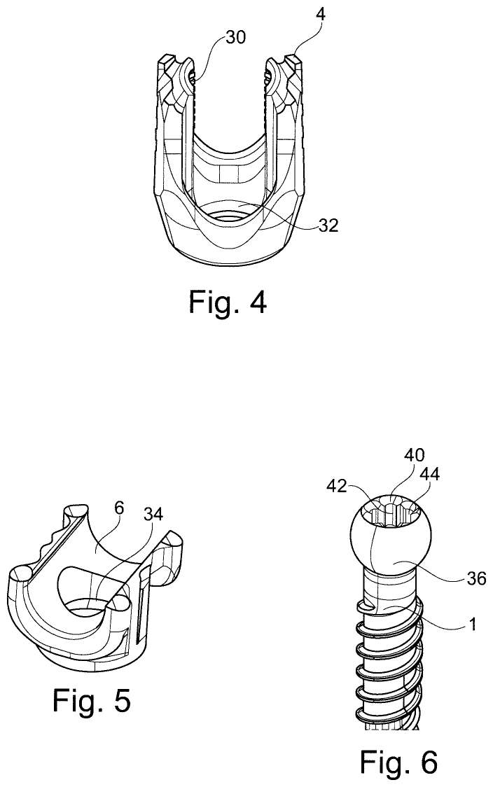

FIG. 4 shows the body (tulip) 4 which can be or is articulated to the screw (pedicle screw) 1. The sleeve-shaped body 4 has a U-shape in side view, i.e. an essentially sleeve-shaped shape with two diametrically opposite longitudinal slots at the proximal sleeve portion and a radial constriction at the distal sleeve end. An internal thread 30 is formed on the side of body 4 facing the tool 2 (in the region of the longitudinally slotted sleeve portion), which is complementary to the external thread 28 of the tool-side threaded part 8, i.e. can receive/accommodate it.

On the distal side of the body 4, i.e. the side facing the screw 1, an internal dome 32 is formed, i.e. a bulbous through-bore. When the body 4 is put over the distal tip of the screw 1 and pushed proximal towards the screw head 36, the internal dome 32 of the body 4 encompasses/grasps the screw 1 at its upper end (spherical screw head 36). Thus, the body 4 is movably held at the upper end (screw head 36) of the screw 1. In the clamped state, i.e. when the external thread 28 of the threaded part 8 is completely screwed into the internal thread 30 of the body 4, the threaded part 8 presses the tool 2 or the tool shank 12 by an axial force against the counterholder 10 into the screw 1 (its blind hole on the screw head side). The abutment is established by the body 4, which is in contact with screw 1 or its screw head.

FIG. 5 shows the insert 6, which can optionally be arranged in the body 4. The insert 6 has an axial projection with an internal bore 34, which fits exactly with the shank portion 14 below the counterholder 10 of the tool 2, but can also be larger, and which can be or is inserted into the internal dome 32 of the body 4 to press against the screw head 36. The counterholder 10 of tool 2 does not touch the insert 6 even when the threaded part 8 is completely screwed in place in the body 4. The insert 6 therefore remains movable in the body 4 even when tool 2 is clamped with the body 4. Therefore, the insert 6 is not in a clamped state with the body 4 when the tool 2 is clamped with the screw 1, if the threaded part 8 is screwed in the body 4, and is therefore movable.

FIG. 6 shows the ball-shaped/spherical screw head 36 of the screw 1 to be implanted. A blind hole is formed in the ball-shaped screw head 36 which extends axially to the screw axis A and opens on the proximal side of the screw head 36. In a distal torque introduction portion 42, the blind hole has at least one radially outwardly projecting indentation/groove 44 and at least one radially inwardly projecting prong/rib/web 46. At the edge of the blind hole, there is a (funnel/cup-shaped) inclined insertion region (chamfer) 41, in which bowl/calotte-shaped indentations/grooves 40 are formed, which preferably line up at the same distance from one another in the circumferential direction. In this way, preferably each indentation/groove 40 in the inclined insertion region 41 represents an individual/separate zone of the inclined insertion region in addition to the at least one groove 44 in the torque introduction portion 42 of the blind hole. In the case of a large number of inclined insertion region areas (indentations) 40, an inclined insertion region area 40 adjoins the respective adjacent one in the circumferential direction, or the inclined insertion region areas 40 are intersected with one another and form a common intersection edge. The intersection edge extends into the interior of a tool receiving area 42 of the blind hole.

The recesses/grooves 40 in the inclined insertion region 41 form force-fit and force-fit areas for the aforementioned semi-conical axial (and radial) projections on the shaft/shank shoulder 16 of the tool 2 of the invention. According to the invention, the inclined insertion region areas 40 form form-fit areas for the centering surface 16 of the tool 2.

The screw head 36 represents the abutment, in other words the contact surface, of the body 4 in the clamped state when the threaded part 8 is screwed in place in the body 4. The alignment takes place via a force fit and form fit between the centering surface 16 or the projections 17 formed there and the inclined insertion region 41 or the indentations 40 formed there.

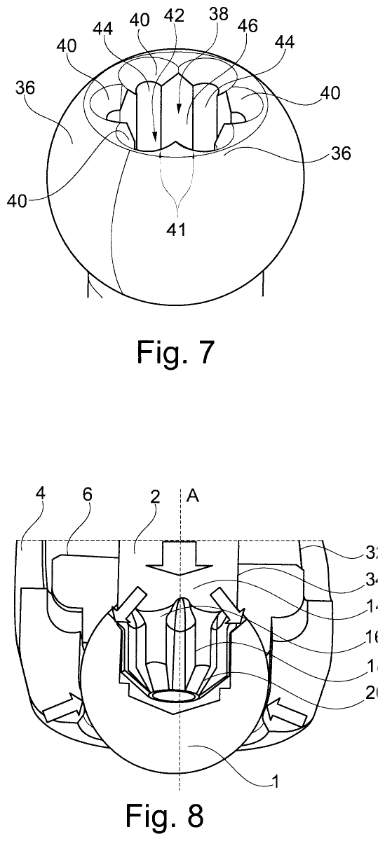

FIG. 7 shows the ball-shaped/spherical screw head 36 in detail. The tool receiving area/torque introduction portion 42 of the screw head 36 or the blind hole 38 has indentations/grooves 44 extending axially toward the screw 1 and machined radially outwards. The radially outwardly pointing indentations/grooves 44 are separated in the circumferential direction by radially inwardly pointing prongs/ribs/webs 46. At the distal edge of the blind hole 38 on the screw head 36, the inclined insertion region 41 is formed. Whereas in conventional screws the inclined insertion region evenly surrounds the entire hole (in the manner of a single lathed chamfer) and projects into the hole like a funnel, the inclined insertion region 41 is designed, so to speak, individually for each indentation/groove 44 in the screw 1 of the present invention, i.e. by circumferentially spaced as well as overlapping, respectively funnel-shaped inclined insertion region areas/indentations 40. This results approximately in the shape of a multi-leaf cloverleaf in plan view.

FIG. 8 shows the tool 2 in the clamped condition according to an advantageous embodiment. The tool 2 is in engagement with the screw 1 and the shank portion 14 underneath/distal to the counterholder 10 is arranged in the internal bore 34 in the insert 6, which in turn is arranged in the body 4. It can be seen from FIG. 8 that the insert 6 still has clearance to the body 4 and is thus movably arranged therein, as seen in the longitudinal axis of tool 2.

The blind hole 38 in the screw 1 or screw head 36 is deeper than the length of the tool portion/torque transmission portion 18 and the adjoining end portion 22 starting from the distal (flattened tool tip) to the shaft/shank portion 16. In other words, the tool 2 in the clamped condition at the flattened tool shaft tip 22 is not in operative engagement/facing abutment with/on the blind hole 38 of the screw 1, whereas the torque transmission region 18 of the tool 2 is in operative engagement with the torque introduction portion 42 of the blind hole 38. This means that the force applied to the screw 1 by the tool 2 is partly (in addition to the torque transmission portion 18) also applied by the projections/centering surfaces 17 in the shaft/shank shoulder 16 to the inclined insertion region 41 or the dome-shaped inclined insertion region areas/indentations 40 formed there.

The indentations or axial grooves 44 in the torque introduction portion 42 of the blind hole 38 each form form-fit and force-fit regions for the ribs/webs 20 in the torque transmission portion 18 of the tool 2, and the (cup-shaped/calotte-shaped) inclined insertion region areas/indentations 40 form foam-fit and force-fit regions for the centering surfaces/spherical projections 17 of the tool 2. The centering surfaces 17 of the tool 2 are designed analogously to the inclined insertion region areas 40 of the tool 2.

In summary, the invention relates to an implantation set for implanting a screw 1 in a human or animal body, comprising a tool 2 and a screw 1 designed to receive the tool 2, wherein the screw (1) has a tool-receiving region (42) into which or onto which a torque transmission region (18) of the tool (2) can be or is inserted placed to transmit torque, wherein at least one inclined insertion region (41) is formed on the tool receiving region (42) to guide the tool (2) during insertion of the screw (1), characterized in that the tool (2) forms at least one centering surface (16), which is matched to the inclined insertion region (41) such that when the centering surface (16) comes into contact with the inclined insertion region (41), a form fit and force fit is created.

1. An implantation set for implanting a screw in a human or animal body, the implantation set comprising:
a tool having a tool shank; and
a screw designed to receive the tool, for which
the screw comprises a tool mount in the form of a blind hole comprising a torque introduction portion into which or onto which a torque transmission region on the distal end region of the tool shank can be or is inserted or placed to transmit torque, wherein the tool mount of the screw proximal relative to the torque introduction portion forms an inclined insertion region for guiding and/or centering the tool shank during inserting it in/placing it on the screw, characterized in wherein
the tool shank forms a centering surface which is proximal relative to the torque transmission portion and matched to the inclined insertion region such that when the centering surface comes into contact with the inclined insertion region, a form fit and preferably a force fit is created in circumferential direction in addition or as an alternative to the form fit between the torque transmission portion and the torque introduction portion.

2. The implantation set according to claim 1, wherein the torque transmission portion on the tool shank of the tool and the torque introduction portion on/in the tool mount of the screw each have at least one or more axially extending ribs or webs which are equally spaced in the circumferential direction and at least one or more axially extending grooves which are equally spaced in the circumferential direction.

3. The implantation set according to claim 1, wherein
the tool shank forms or has a radially protruding shaft/shank ring or shoulder which delimits the torque transmission portion proximally in axial direction and on the distal side of which the centering surface is formed in which a number of protrusions is formed, and
the inclined insertion region, arranged on the proximal end of the torque introduction portion, forms a number of indentations or grooves which protrude in an axial and also in a radial direction and further are of concave design, which can be brought into form-fitting engagement with the protrusions on the centering surface.

4. The implantation according to claim 1, wherein the ribs or webs which extend axially and protrude radially outward on a cylindrical outer surface of a distal shank portion of the tool, forming the torque transmission portion, extend beyond a distal frustoconical outer surface of a tip of the tool in a distal direction.

5. The implantation set according to claim 1, wherein a sleeve-shaped threaded part is placed on the tool shank so as to be movable relative thereto, said threaded part having an external thread at its distal end and a body or tulip being movably supported on the screw, which encompasses a screw head and forms an internal thread at its proximal end portion provided with longitudinal slits, which can be brought into engagement with the external thread of the sleeve-shaped threaded part.

6. The implantation set according to claim 5, wherein a ring-shaped or shaft shoulder type counterholder is formed or fixed on the tool shank and configured and/or adapted to be axially inserted into longitudinal slits in the body/tulip, in order to apply a torque, in particular a restraining torque onto the body/tulip, wherein the sleeve-shaped threaded part is arranged proximal relative to the counterholder such that its external thread is situated on the side of the counterholder and the sleeve-shaped threaded part can be axially moved into contact with the counterholder in a distal direction, if the sleeve-shaped threaded part is screwed into the body/tulip while being restrained by means of the counterholder.

7. A medical tool for screwing in a screw in a human or animal body, the medical tool comprising a torque transmission portion which is formed on the distal end region of a tool shank and configured and/or adapted to be inserted in/placed on a torque introduction portion of a tool mount of the screw, wherein a radially protruding shaft/shank ring or shoulder is formed or arranged on the tool shank, so as to be positioned proximal relative to the torque transmission portion, and delimits the torque transmission portion in a proximal direction, wherein a centering surface is formed on the distal side of the shaft/shank shoulder and comprises or forms a number of protrusions.

8. The tool according to claim 7, wherein the torque transmission portion comprises a number of ribs/webs which are arranged in axial extension relative to the protrusions formed in the centering surface, such that the protrusions formed in the centering surface extend radially beyond the respectively assigned ribs/webs.

9. The tool according to claim 7 further comprising a sleeve-shaped threaded part which is pushed onto the tool shank so as to be rotatable and axially shiftable and can be brought into axial abutment on an annular counterholder which is formed or fixed on the tool shank and has an oval or elliptic cross-section.

10. A pedicle screw comprising a bone screw and a body/tulip encompassing a screw head, in which a blind hole type tool mount is formed, consisting of a torque introduction portion and an inclined insertion region proximally adjoining it in the form of a chamfer circumferentially surrounding the torque introduction portion, wherein a number of indentations is formed in the inclined insertion region which extend outward in axial and radial direction.

11. The pedicle screw according to claim 10, wherein the torque introduction portion comprises a number of axially extending and radially recessed grooves which are and arranged in axial extension relative to the indentations formed in the inclined insertion region, such that the indentations formed in the inclined insertion region extend radially beyond the respectively associated grooves.

12. The pedicle screw according to claim 11, wherein the indentations formed in the inclined insertion region surround the respectively associated grooves in the torque introduction portion in the radial direction of the blind-hole type tool mount forming a funnel.

13. The pedicle screw according to claim 11, wherein the indentations formed in the inclined insertion region have a uniform outer radius which is curved towards outside, which is dimensioned such that these outer radii of the indentations adjoining in the circumferential direction intersect each other.
